(19) United States
(12) Patent Application Publication
(10) Pub. No.: US 2015/0233932 A1
(43) Pub. Date: Aug. 20, 2015

(54) Methods, Systems, and Compositions for Enrichment of Rare Cells

(71) Applicants: Ching-Ping Tseng, Taoyuan (TW); Hung-Chih Lin, New Taipei City (TW)

(72) Inventors: Ching-Ping Tseng, Taoyuan (TW); Hung-Chih Lin, New Taipei City (TW)

(21) Appl. No.: 14/182,710

(22) Filed: Feb. 18, 2014

(51) Int. Cl. G01N 33/574; G01N 1/40

(52) U.S. Cl. CPC G01N 33/57492; G01N 1/405; G01N 2333/70589

(57) ABSTRACT

The present invention discloses a highly efficient method of isolating circulating tumor cells in a blood sample by removing leukocytes and other interfering components in a blood sample. Exemplary isolation method relies on a specially configured separation column for magnetic separation of leukocytes from circulating tumor cells. Also disclosed are systems, devices, and reagents for performing the method, as well as diagnostic methods for early cancer detection, screening, and treatment monitoring utilizing the cell isolation method.

### CROSS-REFERENCE TO RELATED APPLICATIONS

This application claims the benefit of Provisional Application No. 61/766,231, filed on Feb. 19, 2013. The above priority application is hereby incorporated herein by reference.

### FIELD OF THE INVENTION

The invention pertains to the field of cell isolation and enrichment from peripheral blood. More particularly, the invention pertains to a method for the enrichment of rare cells by depleting unwanted cells in a peripheral blood sample. This invention also pertains to devices, systems, and compositions for performing the same.

### BACKGROUND OF THE INVENTION

Various strategies have been developed to enrich and isolate rare cells from peripheral blood. For example, the presence of circulating tumor cells (CTCs) in peripheral blood has been documented since 1869. Based on the difference for the size and density of CTCs and normal blood cells, CTCs can be isolated by membrane filtration or density gradient centrifugation. Antibody recognizing surface antigen on tumor cells also can be used to capture and isolate CTCs in a highly specific manner. The epithelial cell adhesion molecule (EpCAM) is one of the most common surface antigens for the cancer cells with epithelial origin. Accordingly, several CTCs isolation platforms such as CellSearch™ system, Magnetic Activated Cell Sorting System (MACS®), Adna Test, and microfluidic CTC chips have been developed to selectively capture CTCs of epithelial origin from whole blood.

Tumor cells are heterogeneous and not all types of CTCs can be enriched solely by the aforementioned “positive selection” methods. Cancer cells with highly invasive and metastatic nature may undergo epithelial-to-mesenchymal transition (EMT) by which the epithelial tumor cells alter their morphology and down-regulate the expression of epithelial cell markers. As a result, CTCs that are negative for epithelial cell markers are also present in the peripheral blood of cancer patients. In order to enrich all types of CTCs, an approach referred to as negative selection or negative enrichment has been reported. Accordingly, red blood cells are lysed followed by immunodepletion of CD45⁺-leukocytes. Cellular properties of the undepleted cells can then be characterized. Theoretically, all types of CTCs, either epithelial marker positive or negative, can be fully recovered by this method. Negative selection and enrichment of CTCs may therefore represent a better approach for CTCs isolation.

When currently available platforms are used for leukocyte depletion and CTCs enrichment, significant amounts of CD45⁺-leukocytes are co-present with the rare cells such as CTCs. The high-level of left-over CD45⁺-leukocytes presents a real challenge to subsequent analysis of the isolated cells as it significantly interferes with the analysis, thereby, limiting the utility of the isolated cells.

Therefore, there still exists an unmet need to overcome the challenges of enrichment of rare cells such as for clinical detection of CTCs.

### SUMMARY OF THE INVENTION

In view of the above, it is an object of this invention to provide a method for detection of CTCs and rare cells in a sample that is sensitive, accurate, inexpensive and easy to perform.

It is also an object of the present invention to provide reagents and diagnostic kits to enable clinical applications of cell-based molecular diagnostics, particularly in cancer patient care.

It is a further object of the present invention to provide a system for performing cell-based diagnostic assay to detect CTCs or other rare cells in clinical specimens and other biological samples.

The above objects of the present invention are satisfied by the inventors' unexpected discovery of a magnetic separator design and separation protocol that resulted in a highly efficient depletion of unwanted cells in peripheral blood, thereby, overcomes the long-standing problem of leukocyte contamination in isolation of CTCs and rare cells from biological samples.

Accordingly, a first aspect of the present invention is directed to a method for isolating and enriching non-leukocyte nucleated cells in a blood sample. Methods in accordance with this aspect of the present invention will generally include the steps of labeling leukocytes in a nucleated cell suspension of the blood sample with magnetic nanoparticles; loading the cell suspending onto a separation column capable of capturing the magnetically labeled leukocytes to yield a leukocytes-depleted filtrate; collecting the filter; and repeating the labeling, loading, and collecting steps for a predetermined number of cycles or until the amount of leukocytes in the filtrate is below a predetermined level.

Labeling of the leukocytes can be achieved by a number of ways. In a preferred embodiment, anti-CD45 antibody is used to couple the magnetic nanoparticles with the leukocytes. More preferably, a CD45 depletion cocktail comprising tetrameric antibodies directed against CD45 and dextran is used. In this embodiment, the cell suspension is incubated with the CD45 depletion cocktail and then mixed with the dextran-coated magnetic nanoparticles. Other means of specifically attaching magnetic beads to CD45⁺-leukocytes commonly known in the art may also be advantageous used. For example, the anti-CD45 antibody can be conjugated in a single step process to the magnetic beads and incubated with cell suspension to label the leukocytes. Alternatively, an antibody recognizing the Fc region of anti-CD45 antibody is conjugated to the magnetic beads and incubates with cell suspension to label the leukocytes. In another embodiment, cocktails of antibodies against various types of leukocyte surface antigens are used.

In a further preferred embodiment, erythrocytes in the blood sample are removed first prior to the labeling step. Any commonly known methods of removing erythrocytes may be use. For example, a red blood cell (RBC) lysis buffer may be used to break up the erythrocytes, followed by centrifugation to separate nucleated cells from the erythrocyte. The pellet can then be washed and re-suspended in a cell culture medium to form a nucleated cell-suspension. Examples of cell lysis buffer may include but not limited to a buffer solution containing 0.15 mol/l NH₄Cl and 10 mmol/l NaHCO₃.

While the magnetic nanoparticles are preferably coated with dextran to facilitate attachment to the ligands or cells, other attachment schemes may also be advantageously used. For example, streptavidin-biotin pairs may be used in place of dextran-tetrameric antibody pair. In this embodiment, nano- or micro-magnetic particles can be coated and conjugated with streptavidin and through streptavidin-biotin interaction, links biotin-labeled CD45 antibody to the nano- or micro-magnetic particles. Any other functional pairs of interaction molecules can be used to facilitate the conjugation of antibody to the magnetic particles.

Separation columns suitable for the present invention will preferably have a body in the shape of a hollow cylinder with an entry opening on one end and an exit opening on the other end, and a cylindrical hollow space connecting the entry opening and the exit opening to form a passage channel.

The body of the separation column can be made of any number of materials, so long as it does not react with the cell suspension and magnetic particles. It should also not interfere with any externally applied magnetic field. In a preferred embodiment, the separation columns are made of plastics or glass.

The passage channel is packed with spherical separation beads. The beads are evenly distributed to fill up the channel. The body may further include a barrier at the exit end to hold the separation beads but allow cells and fluids to flow through.

The separation beads are preferably made of a ferromagnetic material and have a uniform size of about 0.5 mm to about 1.5 mm. In a preferred embodiment, the beads are 1.0 mm in size. Exemplary ferromagnetic material may include but not limited to Fe, Co, Ni, Gd, Dy, CrO2, MnAs, MnBi, EuO, ferrite, and Y₃Fe₅O₁₂. The beads are also preferably coated with an anti-corrosion coating. In a preferred embodiment, the beads are made of iron coated with nickel.

When the column is packed with the separation beads and placed in a magnetic field, the beads become magnetized to attract the magnetic nanoparticles in the cell-suspension. As the nanoparticles are tethered to leukocytes, the leukocytes are effectively pulled out of the suspension, while non-labeled cells are allowed to pass through the column unimpeded.

It is an unexpected discovery of the present invention that when the separation column is configured such that the separation beads are packed in the column to a packing density of about 963 beads/cm³, and placed in a circular magnetic field, a surprisingly high separation efficiency is achieved. In this preferred configuration, the ratio of surface area to void volume is about 61 cm⁻¹. While not intending to be bound by any particular theory, we believe that this ratio results in a space between the separation beads larger than 10 cells diameter, which presumably improves cell separation efficiency and retains cell viability by minimizing shear stress and cell damage. Thus, in a preferred configuration, the combination of a separation column packed with uniform sized spherical separation beads made of iron with a size of about 1.0 mm and magnetic nanoparticles with a uniform size of about 200 nm activated by a circular magnetic field results in surprisingly good nanoparticle capture, which in turn, translates into higher leukocyte depletion efficiency in the methods of the present invention.

It is yet another unexpected discovery of the present invention that the use of cell culture medium in the depletion process for washing and suspending the cells resulted in improved cell viability. This is in stark contrast to most prior art protocols which recommend the use of PBS in cell suspension and washing. In the present invention, we surprisingly discovered that PBS is not conducive to maintaining cell viability during the depletion process. Thus, in methods described herein, culture media or other nutrition-containing medium should be used for washing and suspending cells. Preferably, the culture medium is one selected from DMEM, RPMI-1640, F12, MEM or other nutrition-containing medium in the presence of appropriate percentage of fetal bovine serum in the range of 1%-20%.

Magnets suitable for the present invention can either be a permanent magnet or an electromagnet, so long as it is capable of generation a circular magnetic field to magnetize the separation beads in the column. Exemplary magnets are the EasySep column (Stem cell Technologies). It will be understood by those skilled in the art that the strength of the magnetic field has to be sufficient to capture the nano- or micro-magnetic particles on the separation beads to facilitate the retention of leukocytes in the separation column that is filled up with separation beads.

Finally, the depletion process may be repeated to further improve depletion ratio. As used herein, repeating the process means that the end result of each depletion run (i.e. the filtrate that came through the column) is used as the starting material in the next round of depletion. Preferably, the process is repeated between 3-4 times to maintain an optimal ratio of depletion of leukocytes and retention of non-leukocyte nucleated cells.

A second aspect of the present invention is directed to a system for isolating and enriching non-leukocyte nucleated cells in a blood sample. Systems in accordance with this aspect of the invention will generally include a separation column having a hollow body with an entry opening on one end, an exit opening on the other end, and a cylindrical hollow space connecting the entry opening and the exit opening to form a passage channel therethrough. A plurality of spherical separation beads is disposed in the passage channel of the separation column. The separation beads are preferably comprised of a ferromagnetic material coated with an anti-corrosion coating, have a uniform size of about 0.5 mm to about 1.5 mm, and are capable of being magnetized to capture a cell labeled with a dextran-coated magnetic nanoparticle having a uniform size of about 200 nm. The system also includes a magnet capable of generating a circular magnetic field.

In operation, the separation beads are packed in the separation column to fill the passage channel, the magnetic nanoparticles are attached to leukocytes with surface antigen such as CD45⁺ in a blood sample to be passed through the separation column, and the separation column is placed in the circular magnetic field generated by the magnet such that the separation beads are sufficiently magnetized to magnetically capture the nanoparticles.

The structural details of the column, separation beads, magnetic nanoparticle, and magnet are as described above. The system may either be configured as a manually operated system, a semi-automated system, or a fully automated system.

For example, in one embodiment, the column, magnets, separation beads are provided separately to be assembled manually at run time. In another embodiment, actuators and control computers are included to automate operation of the components.

A third aspect of the present invention is directed to a method of early detection of cancer in a patient. Methods in accordance with this aspect of the invention will generally include the steps of obtaining a blood sample from the patient; depleting leukocytes in the blood sample to yield a leukocyte-depleted cell-suspension; enumerating circulating tumor cells (CTC) in the leukocyte-depleted cell-suspension; and then determining a diagnosis based on the result of the enumeration step.

In this aspect of the invention, depletion of leukocyte is preferably removed or depleted by performing a method as described in the first aspect above. Depending on the definition of CTC used, different diagnostic information may be obtained from the resulting CTC count. For example, as a generally screening assay agnostic as to the particular type of cancer, CTC may preferably defined as nucleated cells in the leukocyte-depleted cell-suspension that are EpCAM⁻CD45⁻ or EpCAM⁺. In this embodiment, a diagnosis for high cancer risk is determined if the amount of EpCAM⁻CD45⁻ cells is >650 cells/ml or the amount of EpCAM+ cells is ≧5 cells/ml.

In another embodiment, the CTC-based assay may be used for determining the prognosis of a patient suffering from head and neck squamous cell sarcoma. Assays in accordance with this embodiment will define CTC as cells that are positive for PDPN and/or EpCAM. A diagnosis may be determined by computing a ratio for the number of CTC expressing PDPN to the total number of CTC, wherein if the ratio is greater than 20%, the patient is said to have a poor prognosis for 6-months survival after chemotherapy.

A fourth aspect of the present invention is directed to a CTC-based screening and early detection assay for identifying an asymptomatic subject who is at risk of infiltrative papillary thyroid microcarcinoma (IPTM). Assays in accordance with this aspect of the invention will generally include the steps of obtaining a blood sample from the subject; depleting leukocytes in the blood sample to yield a leukocyte-depleted cell-suspension; enumerating the number of CTC in the leukocyte-depleted cell-suspension, wherein CTC is defined as EpCAM⁺ cells; determining a diagnosis based on the level of CTC present, wherein if the CTC count is above a predetermined level, a likelihood of IPTM is indicated.

In another embodiment, the assay may be used to detect papillary thyroid microcarcinoma (IPTM). In this embodiment, CTC is defined simply as EpCAM⁺ cells in the leukocyte-depleted cell-suspension. Normally, the CTC counts in this assay for healthy subjects are expected to be about 5 cells/ml. Thus, a CTC count higher than 5 cells/ml may be a warning sign requiring further attention. However, the threshold level of CTC may be chosen to reflect a different desired level of assay sensitivity. For example, patients who have CTC levels as high as 500 cells/ml are still asymptomatic, hence, a threshold level may be set at 500 cells/ml. Asymptomatic patients after treatment have seen their CTC level drop to around 200 cells/ml, hence, warning threshold level may also be set at 200 cells/ml.

A fourth aspect of the present invention is directed to a method of monitoring or assessing the effectiveness of a cancer treatment on a patient. As described above, various diagnostic information may be obtained by enumerating CTC in the leukocyte-depleted cell-suspension. When the level of CTC before a cancer treatment is compared to the level of CTC after the treatment, the comparison may serve as a benchmark to assess whether the particular treatment method is effective on the particular patient. Monitoring or assessment may be done by single point comparison or by taking a time-series of the CTC level and then analyzing the trend to make a diagnostic determination.

A fifth aspect of the present invention is directed to a kit for performing CTC-based assays. Kits in accordance with this aspect of the invention are designed to facilitate performance of the cell isolation method and CTC-based assays as described above. Thus, they generally include reagents required for performing the cell isolation method and/or CTC enumeration step of the assays. They may also include containers, vials, and other tools for facilitating the manipulation of reagents and blood samples. Exemplary tools that may be included in the kit may include but are not limited to needles, blood collection vials, RBC lysis solution and reagent, depletion device and reagents (including but not limiting to separation beads, separation column, anti-CD45 depletion reagents, and nutrition medium in the presence of 1-20% FBS), enumeration reagents (including but not limiting to anti-CD45 antibody, anti-EpCAM antibody, anti-PDPN antibody, anti-thyroid hormone receptor antibody, fluorescence-conjugated secondary antibodies, and DNA binding dye).

Preferably, kits of the present invention will also include an instruction insert with instructions for performing the isolation method or assays as described above. The instruction insert may be in any human understandable format, including but not limited to a written booklet, an instructional DVD, an audio recording, and a printed link to an instructional website.

Other aspects and advantages of the invention will be apparent from the following description and the appended claims.

### BRIEF DESCRIPTION OF THE DRAWINGS

FIG. 1 shows a schematic representation of an exemplary system in accordance with embodiments of the present invention. (A) Exemplary design of PowerMag, as described in Example 1, with separation beads packed into the leukocyte depletion column. (B) Illustrate an exemplary capturing of leukocyte which is labeled with antibody-nanoparticle complex that are magnetically captured by the separation beads.

FIG. 2 shows evaluation of CTC recovery rate by spiking test. (A)-(B) PC3 cells pre-labeled with calcein red/orange dye were spiked into whole blood to prepare blood samples with different PC3 cell concentrations (25, 100, 250, 500, and 1000 cells/ml). After leukocyte depletion by PowerMag, the cells in the filtrate were analyzed by immunofluorescence staining of calcein red/orange, Alexa Fluor 488 and Hoechst 33342 (Panel A). The number and the percentage of cell recovery were determined (Panel B). One-way ANOVA analysis was performed for statistical analysis of cancer cell recovery rate among all groups of cell spiking experiments. The data represent the mean±SE of 3-4 independent experiments.

FIG. 3 shows cell population and proliferative capability of the nucleated cells enriched by PowerMag. (A) PC3 cells were spiked into whole blood and leukocyte depletion was performed by PowerMag. The leukocyte-depleted cell filtrate was analyzed by immunofluorescence staining using anti-EpCAM (green) and anti-CD45 (red) antibodies. Leukocytes were used as a positive control (PC) for immunofluorescence staining of CD45 surface marker. Two major cell populations were defined according to the immunofluorescence staining patterns. (B) PC3 cells that were recovered by PowerMag were cultured for 1-5 days. A representative PC3 cell colony at the indicated days of culture was shown (upper panel). At day 5 of culture, the cells were labeled with calcein red/orange and were analyzed by immunofluorescence staining using the anti-EpCAM antibody and Hoechst DNA staining dye.

FIG. 4 shows the population and cell size of CTCs isolated from healthy donors and cancer patients. (A) Peripheral blood from healthy donors and cancer patients was processed by PowerMag for depletion of leukocytes. The enriched nucleated cells in the cell filtrate were analyzed by immunofluorescence staining using anti-EpCAM (green) and anti-CD45 (red) antibodies. Two major cell populations, EpCAM⁺CD45⁻ (EpCAM⁺-CTCs) and EpCAM⁻CD45⁻, were defined. Fifteen representative images for each cell population were shown. Note the heterogeneous distribution for the size of EpCAM⁻CD45⁻ cells isolated from cancer patients. (B) The diameter of EpCAM⁺-CTCs cells (n=150) was determined and the cell size distribution was plotted. (C) EpCAM⁺CD45⁻ (EpCAM⁺-CTCs) and EpCAM⁻CD45⁻ cells isolated from cancer patients were analyzed by vital staining with calcein red/orange dye. A representative field was shown.

FIG. 5 shows the number of EpCAM⁺CD45⁻ and EpCAM⁻CD45⁻ cells in healthy control and cancer patients. (A-B) Blood samples from healthy donors and the patients with CRC or HNSCC collected at baseline were analyzed by PowerMag. The number of EpCAM⁺CD45⁻ (panel A) and EpCAM⁻CD45⁻ (panel B) cells for each individual was plotted. Mediums are indicated by horizontal lines.

### DETAILED DESCRIPTION

To further illustrate the present invention, the following specific embodiment and illustrative examples are provided. Although the present invention will be further described in terms of specific exemplary embodiments and examples below, it will be appreciated that the embodiments disclosed herein are for illustrative purposes only and various modifications and alterations might be made by those skilled in the art without departing from the spirit and scope of the invention as set forth in the appended claims.

### Example 1

### A Negative Selection System for Effective Leukocyte Depletion and Enhanced Detection of EpCAM Positive and Negative Circulating Tumor Cells

### Materials

The anti-CD45 depletion kit was purchased from StemCell Technologies (Vancouver, BC). The anti-EpCAMand phycoerythrin (PE)-labeled anti-CD45 antibodies were from Abcam (Cambridge, England). The vacutainer tube with K3EDTA was from BD Biosciences (San Diego, Calif.). The Alexa Fluor 488-conjugated donkey anti-mouse IgG (H+L), the CellTrace™ calcein red/orange AM and the Hoechst 33342 staining dye were from Invitrogen (Carlsbad, Calif.).

### Patients and Blood Collection

This study was approved by the Institute Review Board of Chang Gung Memorial Hospital with the approval ID of 99-4095B and 100-4623C. The peripheral blood (4 ml/test) was withdrawn from 27 healthy donors and 52 cancer patients with written informed consent. The patients with locally advanced carcinoma and distant metastasis were treated with standard concurrent chemoradiotherapy and palliative chemotherapy, respectively. Chemotherapy regimens were given based on the cancer type of the patients; bevacizumab plus FOLFIRI or FOLFOX for CRC and cisplatin with or without 5-flourouracil for HNSCC patients. The baseline was defined as the CTC level before receiving the first dose of chemotherapy. Blood collection for the baseline and the first follow-up was performed at a time interval of 2 weeks.

### System Design

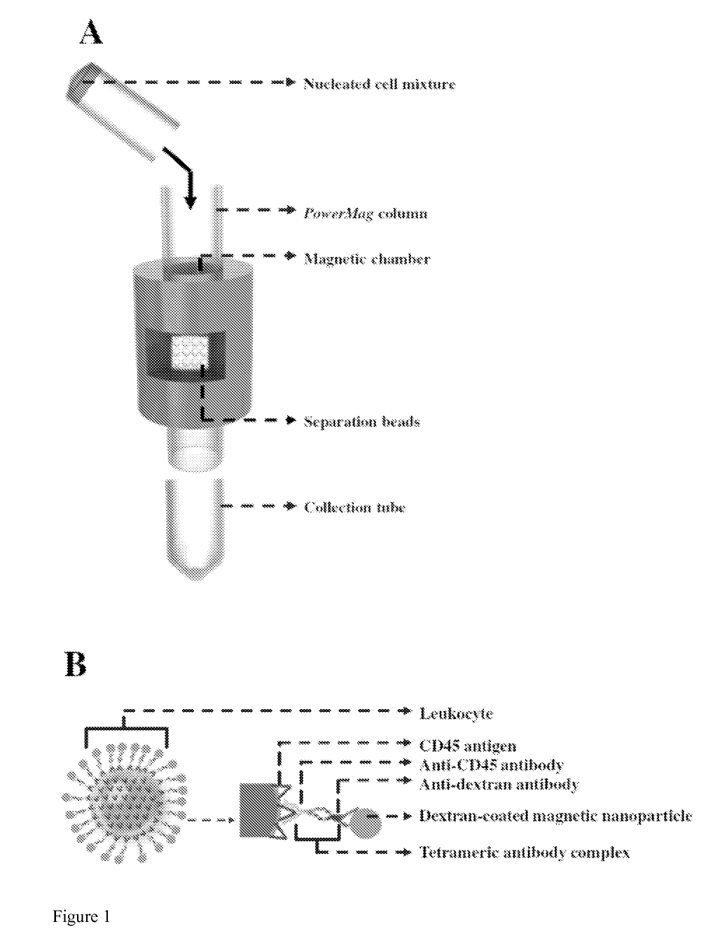

The exemplary system disclosed herein (henceforth referred to as PowerMag) consists of a custom-made magnetic chamber and a separation bead-packed plastic column. The stainless separation beads with the size of 0.4 mm³ distributed evenly and filled up the space of the column (FIG. 1A). When exposed to magnetic field, the separation beads became magnetized that allowed the binding of magnetic nanoparticles in the following CTC isolation process. The loading density for the separation beads was 963 beads cm⁻³. The surface area to void volume ratio was 61 cm⁻¹ which is larger than such ratio reported in prior art. The increase of the column in the surface area to void volume ratio potentially improves cell separation efficiency and retains cell viability when cells pass through the column.

### Enrichment and Isolation of Circulating Tumor Cells

For negative selection of CTCs by methods and systems of the present invention, erythrocytes were lysed by mixing whole blood (4 ml) with RBC lysis buffer (0.15 mol/l NH₄Cl and 10 mmol/l NaHCO₃) for 5 min at room temperature. The mixture was then centrifuged (400×g) at 10° C. for 10 min. The cell pellets were washed and subsequently resuspended in 2 ml of cell culture medium to obtain all nucleated cells. To deplete CD45⁺-leukocytes, CD45 depletion cocktail was mixed with the collected nucleated cells and incubated at room temperature for 15 min. The dextran-coated magnetic nanoparticles were then added to the reaction mixture and incubated at room temperature for 10 min to label CD45⁺-leukocytes (FIG. 1B). The sample was then loaded into the column to separate CD45⁺-leukocytes from the other nucleated cells. During the process, CD45⁺-leukocytes were captured on the surface of separation beads, leaving CD45 cells to pass through the column. The cell filtrate was then centrifuged (400×g) for 10 min at 10° C. The pelleted cells were mixed with the CD45 depletion cocktail and the depletion process was repeated for a total of 3-4 times. CTC isolation by this method can be completed within 3 h.

Enrichment of CTCs by EasySep platform was performed as described by the manufacturer (StemCell Technologies) with minor modification. Briefly, after RBC lysis, the nucleated cells were mixed with CD45 depletion cocktail. After adding the magnetic nanoparticles, the reaction tube was placed into a magnetic chamber for 10 min. During the process of leukocyte depletion, CD45+-leukocytes were captured on the surface of separation beads, leaving CD45− cells to pass through the column. The cell filtrate was then centrifuged (400×g, 10 min, 10° C.) and the pelleted cells were mixed with the CD45 depletion cocktail to repeat the depletion process if required.

### Immunofluorescence Staining

For immunofluorescence staining, PC3 cells or leukocyte depleted cell filtrate was incubated with anti-EpCAM antibody and the Hoechst 33342 DNA staining dye at room temperature for 1 h. After several washes and centrifugation, Alexa Fluor 488-conjugated donkey anti-mouse antibody was added to the cell suspension and kept in the dark for 40 min. When indicated, the cells were incubated with PE-labeled anti-CD45 antibody to differentiate CD45⁺-leukocytes from other nucleated cells. After removal of the unbound antibody, immunofluorescent images were captured and analyzed by fluorescence microscopy (Zeiss Axiovert 200 M).

### Statistical Analysis

One-way ANOVA (SPSS, ver. 10.0.7C) was performed for statistical analysis of cancer cell recovery rate. Analyses of the other data were performed by Student's t-test. A p<0.05 was considered statistically significant.

### Results

### Leukocyte Depletion Efficiency and CTC Recovery Rate

In this example, an exemplary system, PowerMag, was designed to improve the efficiency of leukocyte depletion and to enrich CTCs by a negative selection scheme. We first compared our system against EasySep (StemCell Technologies) for efficiency in the depletion of CD45⁺-leukocytes. EasySep is currently considered the benchmark system for cell separation. Leukocytes (10⁶ or 10⁷) from healthy donors were processed according to the protocols of PowerMag and EasySep. The number of CD45⁺-leukocytes that remained in the leukocyte-depleted cell filtrate was determined. As expected, the amounts of CD45⁺-leukocytes that remained in the cell filtrate decreased with each cycle of depletion by either PowerMag or EasySep (Table 1). However, the efficiency of leukocyte depletion is significantly different. When 10⁶ and 10⁷ leukocytes were depleted by PowerMag for 4 times, the number of CD45⁺-leukocytes that remained in the cell filtrate was 10±1 (n=3) and 20±2 (n=3), respectively. After leukocyte depletion by EasySep for 4 times, the number of CD45⁺-leukocytes that remained in the cell filtrate was 103±9 (n=3) and 126±16 (n=3) when the assay started with 10⁶ and 10⁷ leukocytes, respectively. These data imply that PowerMag is more efficient than EasySep in filtering out CD45⁺-leukocytes.

The efficiency for PowerMag and EasySep in the recovery of CTCs was also compared. One hundred prostate cancer PC3 cells were pre-labeled with calcein red/orange AM for cell tracking and were spiked into the cell suspension containing 10⁶ or 10⁷ leukocytes. After leukocyte depletion by PowerMag and EasySep, the number of PC3 cells that were recovered in the cell filtrate was determined. The number of PC3 cells that remained in the cell filtrate was decreased when the number of depletion by PowerMag or EasySep was increased (Table 1). After leukocyte depletion by PowerMag for 4 times, 77.7±2.7% (n=3) and 78.4±3.6% (n=3) of the spiked PC3 cells were recovered from the cell suspension containing 10⁶ and 10⁷ leukocytes, respectively (Table 1). On the other hand, 39.0±2.9% (n=3) and 48.4±5.9% (n=3) of the spiked PC3 cells were recovered by EasySep from the cell suspension with 10⁶ and 10⁷ leukocytes, respectively. These data imply that PowerMag is more efficient than EasySep in the recovery of CTCs.

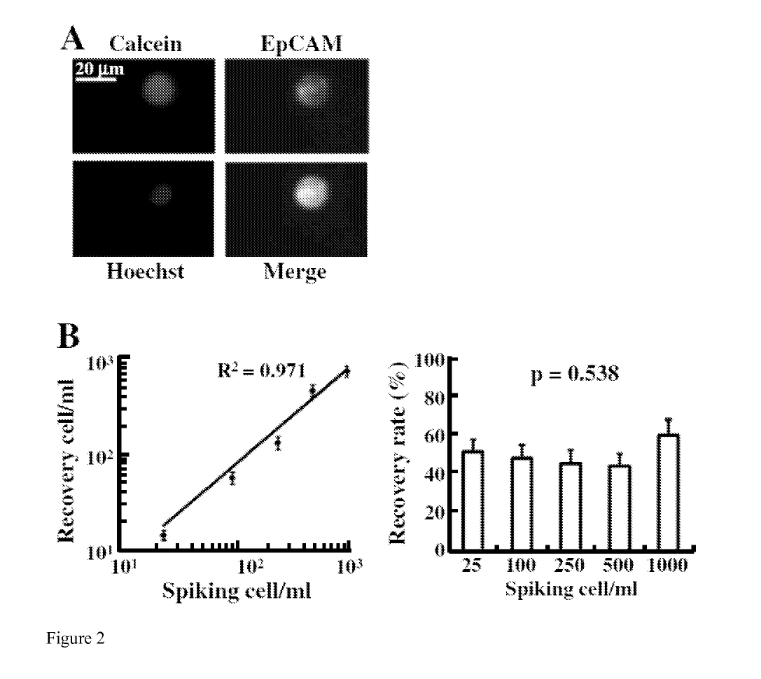

To evaluate whether there is a correlation between the number of cancer cells in the blood and the number of cancer cells recovered by PowerMag, PC3 cells were pre-labeled with calcein red/orange and were spiked into whole blood to make up blood samples with different concentrations of PC3 cells (25, 100, 250, 500 and 1000 cells/ml). The spiked PC3 cells were recovered from the blood by PowerMag and were identified by positive staining of calcein red/orange, EpCAM, and Hoechst 33342 (FIG. 2A). The recovery rates of PC3 cells from the whole blood with 25, 100, 250, 500 and 1000 PC3 cancer cells/ml were 53.4±6.1% (n=4), 49.8±7.4% (n=4), 47.1±7.2% (n=3), 45.8±6.4% (n=3), and 61.9±8.0% (n=4), respectively (p=0.538). The correlation coefficient was 0.971 (FIG. 2B).

In general, only few CTCs are present in the peripheral blood of cancer patients. To evaluate whether PowerMag has the capability to isolate rare amounts of CTCs from blood, PC3 cells were spiked into whole blood at the concentrations of 1, 5 and 10 cells/ml. A total of ten independent experiments were performed to determine the efficiency of PowerMag in the recovery of CTCs. When one PC3 cell was spiked into whole blood, the single PC3 cell can be recovered from the blood in 6 of the 10 independent assays (Table 2). Moreover, an average of 3.0±0.3 (n=10) and 6.7±0.3 (n=10) cells was recovered when 5 and 10 PC3 cells were spiked into whole blood, respectively. PowerMag therefore is able to isolate rare cancer cells from whole blood and is suitable for CTC detection.

### PowerMag for CTC Isolation: Evaluation of Cell Population and Proliferative Capacity

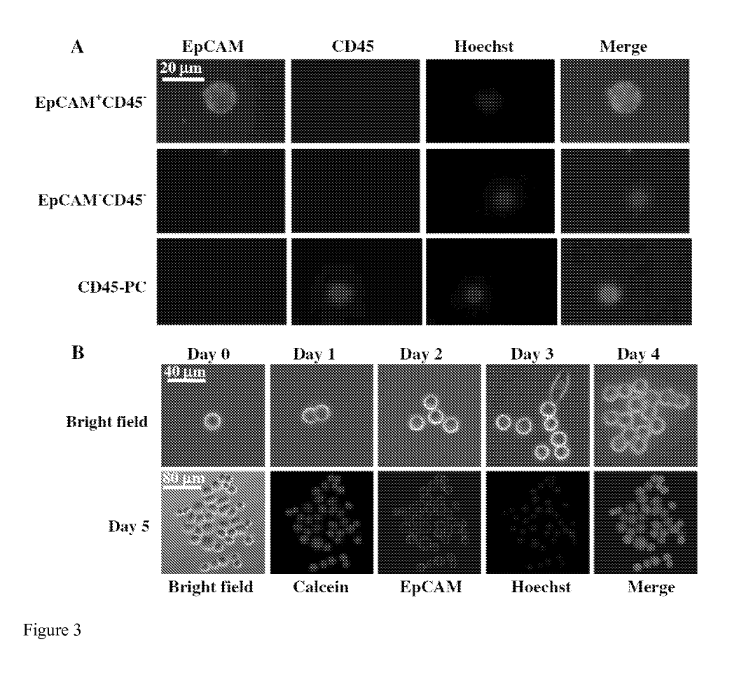

The cell populations that were not depleted by PowerMag were characterized in the following experiment. Unlabeled PC3 cells were spiked into the whole blood from healthy donors. After being processed by PowerMag, the nucleated cells (blue by Hoechst 33342 staining) in leukocyte-depleted cell filtrate were analyzed by immunofluorescence staining using anti-EpCAM (green) and anti-CD45 (red) antibodies. Most of the leukocytes were depleted by PowerMag. Usually less than 5 cells in the cell filtrate were CD45 positive (FIG. 3A). Two other major nucleated cell populations that were EpCAM⁺CD45⁻ and EpCAM⁻CD45⁻ were also identified (FIG. 3A). The EpCAM⁺CD45⁻-cells represented the recovered PC3 cells and were considered as EpCAM⁺-CTCs when clinical samples were analyzed. The second cell population was EpCAM⁻CD45⁻ that was referred to as marker negative cells.

The viability and proliferative capacity of the recovered PC3 cells were characterized by culturing the cells on a Petri dish for up to 5 days. The recovered cancer cells can proliferate and form colonies as observed by phase contrast and fluorescence microscopy (FIG. 3B). These data imply that PowerMag is suitable for isolating viable cancer cells with proliferative capability.

### PowerMag Facilitates Cancer Patient CTC Detection

The suitability of PowerMag in monitoring cancer patient CTCs was assessed by analyzing the blood samples from 24 CRC and 28 HNSCC patients (Table 3). Based on 95.4% confident interval (mean±2 SD) for the number of EpCAM⁺CD45⁺ and EpCAM⁻CD45⁻ cells in the blood samples of 27 healthy donors, the cut-off values for EpCAM⁺CD45⁺ and EpCAM⁻CD45⁻ cells were set to distinguish positive and negative CTC test. Accordingly, the test was considered CTC positive when EpCAM⁺CD45⁻≧5 cells/ml or when EpCAM⁻CD45≧650 cells/ml (Table 4).

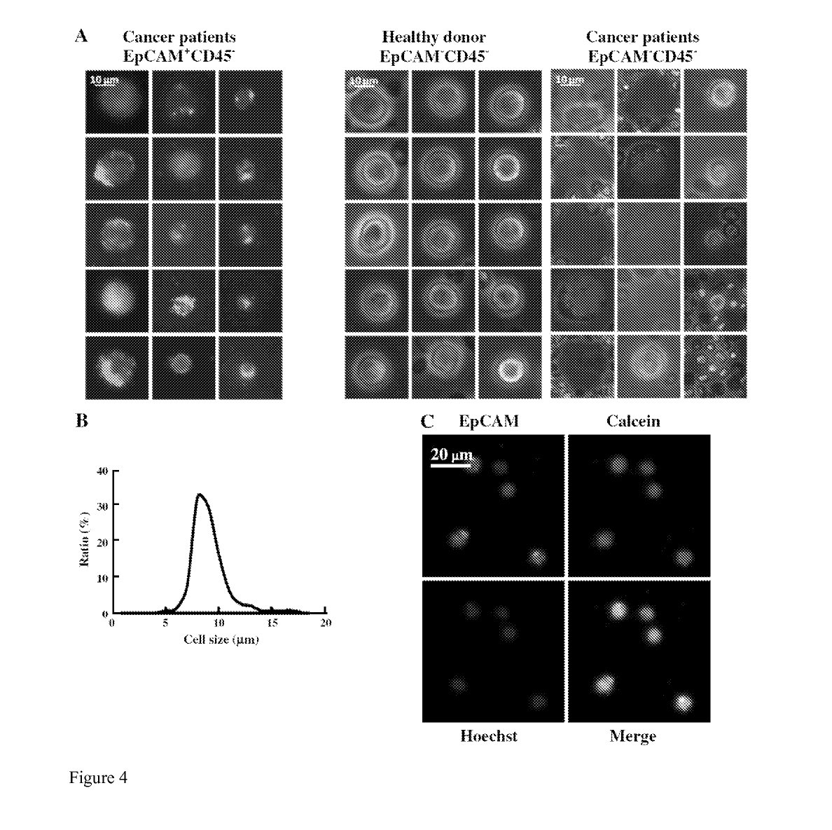

Analyses of CRC and HNSCC blood samples collected at baseline and follow-up after chemotherapy (n=93) revealed that both cell types of EpCAM⁺CD45⁻ and EpCAM⁻CD45⁻ were present in the blood of cancer patients (FIG. 4A). The diameter of EpCAM⁺CD45⁻ cells ranged from 5 to 20 μm with a mean diameter of 9.1±1.7 μm (FIGS. 4A and B). Furthermore, EpCAM⁻CD45⁻ cells obtained from cancer patients were more heterogeneous when compared to the healthy control (FIG. 4A). Some of the cells displayed an unusually large size with the cell diameter reaching 20-70 μm. CTCs isolated by PowerMag were viable and can uptake the calcein orange/red vital staining dye (FIG. 4C).

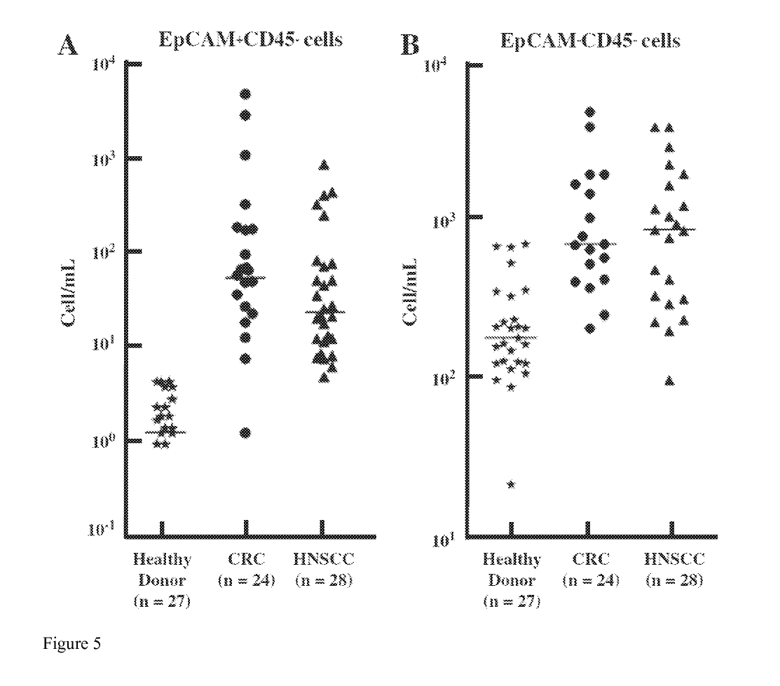

The number of EpCAM⁺CD45⁻ and EpCAM⁻CD45⁻ cells for the blood samples collected at baseline distributed widely for 2-4 log₁₀ (FIGS. 5A and B). Overall, 83.3% of CRC and 100% of HNSCC patients were positive for EpCAM⁺CD45⁻, while 61.1% of CRC and 60.7% of HNSCC patients were positive for EpCAM⁻CD45⁻ (Table 4). When either EpCAM⁺CD45⁻ or EpCAM⁻CD45⁻ was positive, the overall positive rates were 95.8% and 100% for CRC and HNSCC patients, respectively (Table 4). When comparing the blood samples collected at baseline and the first follow-up, the number of EpCAM⁺CD45⁻ and EpCAM⁻CD45⁻ cells was decreased more prominently in the patients with CRC (median: −87.3%; mean: −96.2%) and HNSCC (median: −58.3%; mean: −43.5%), respectively. PowerMag thereby can be used to monitor CTC status of cancer patients.

### Discussion

Isolation and characterization of CTCs hold tremendous potentials for probing the biological insights underlying cancer metastasis, detection of cancer-related gene mutation and discovery of novel cancer biomarkers. In the present study, an exemplary system of the present invention PowerMag is established for efficient leukocyte depletion and isolation of label-free and viable CTCs that provides a new avenue for monitoring cancer progression and treatment response of cancer patients.

A number of devices have been designed to facilitate CTC detection. For example, CellSearch™ system is widely utilized for prognosis evaluation in metastatic breast, colorectal, and prostate cancer. More recently, microfluidic devices have been developed to enrich CTCs from peripheral blood of cancer patients. Several immuno-based methods for positive and negative selection of CTCs have also been reported. The PowerMag system disclosed herein has several significant technical and analytical advantages over prior art systems. First, the separation beads that are used to compose the PowerMag leukocyte depletion column can be reused for hundreds of times without losing the capability to bind antibody-coated nanoparticles. This type of design allows cost-effective CTC isolation. In addition, effective leukocyte depletion is important when marker-negative (i.e. EpCAM⁻CD45⁻) cells are crucial for CTC analysis. Through trial and experimentation, the inventors discovered that the particular sizes of the separation beads used in the PowerMag separation column exhibited an excellent ability to capture magnetically labeled cells while maintaining viability of the unlabeled cells, presumably due to the increased surface area for leukocyte depletion.

According to the data disclosed herein, increasing the depletion cycle from 2 to 4 resulted in an additional 92-94% decrease of CD45⁺ cells while cancer cell recovery was decreased for merely 13-17%. Hence, repeating the depletion process for 4 times is able to achieve maximal depletion of CD45⁻ cells with sufficient recovery of cancer cells. Even so, less than 20 CD45⁺ cells still remained in the leukocyte-depleted cell filtrate. These could be weak CD45⁻ cells that cannot be removed even after being processed by PowerMag for 4 times. In accordance with this notion, few CD45⁺ cells were still visible by immunofluorescence staining of the leukocyte-depleted cell filtrate. These cells were excluded from CTCs and marker negative cell counts. It is therefore demonstrated herein that the PowerMag platform is more efficient compared to all existing prior art platforms for negative selection of CTCs.

The design of PowerMag also allows cancer cells to encounter minimal shear stress when passing through the column. Therefore, CTCs can be recovered more efficiently for subsequent analyses. The recovery rates of CTCs are 77-82% and 46-62% when cancer cells were enriched from leukocyte suspension and whole blood, respectively, that is either better or equivalent to the currently available methods. Last but not the least, PowerMag allows label-free isolation of CTCs that are viable and have the potential to grow. This could open up a promising route to realize personalized cancer therapy. PowerMag thereby provides technical and analytical advantages for CTC detection and isolation.

The threshold values for distinguishing positive or negative CTC test are usually dependent on the specific analytical platforms. When CellSearch system is used for CTC analysis, more than 5 CTCs in 7.5 ml whole blood are considered to have a negative impact on overall survival of cancer patients. For herringbone chip, the test is considered significant when the number of CTCs≧10 cells/ml. Therefore, it is essential to define the reference value for significant CTC count when PowerMag is used to monitor CTC status of cancer patients. In this invention, we integrated our analysis by taking into consideration both EpCAM⁺CD45⁻ and EpCAM⁻CD45⁻ cell populations. Based on 95.4% confident interval (mean±2 SD) for the number of EpCAM⁺CD45⁻ and EpCAM⁻CD45⁻ cells in healthy control, the cell counts are considered clinically significant when the number of EpCAM⁺-CTCs≧5 cells/ml or when the number of EpCAM⁻CD45⁻≧650 cells/ml.

The potential use of PowerMag in monitoring CTC status of cancer patients was also evaluated in this study. By analyzing 93 blood samples obtained from 52 cancer patients, several intriguing observations were revealed. Despite that epithelial cell markers negative CTCs (i.e. EpCAM⁻CD45⁻) have been described previously, no study reports yet have been reported to characterize this cell population. In this study, we found that EpCAM⁻CD45⁻ cells are present in the healthy control as well as cancer patients. However, the number of EpCAM⁻CD45⁻ cells is usually higher in cancer patients when compared to the healthy control. Moreover, EpCAM⁻CD45⁻ cells from cancer patients are typically heterogeneous with some of the cells displaying unusually large size. While only portion of these cells were positive for the surface markers CD146 (endothelial cell marker), CD34 (stem cell and endothelial progenitor cell marker) and pan CKs (epithelial cell marker), the origin of these marker-negative cells (EpCAM⁻CD45⁻) is still unknown. Further investigation is required to characterize the properties of EpCAM⁻CD45⁻ cells. We postulate that EpCAM⁻CD45⁻ cells from cancer patients are a mixture of EpCAM⁻-CTCs and different types of host cells in response to cancer progression and/or therapy. It is contemplated that the number of EpCAM⁻CD45⁻ cells in the peripheral blood may provide an additional indicator to evaluate CTC status of cancer patients.

Similar to other CTC studies, the number of EpCAM⁺-CTCs cells as revealed by PowerMag is correlated with clinical status of cancer patients as determined by standard imaging studies such as computed tomography or magnetic resonance imaging scan. Our data indicate that PowerMag is suitable for monitoring cancer patient CTCs. Notably, from baseline to the first follow-up, the number of EpCAM⁺-CTCs and EpCAM⁻CD45⁻ cells is decreased more prominently in CRC and HNSCC patients, respectively (Table 4). To the best of our knowledge, this is the first study demonstrating the discrepancy between EpCAM-positive and marker-negative cells in different cancer groups. Although the reasons for this discrepancy are not clear, it is likely that CRC which is a type of adenocarcinoma and HNSCC which is a type of squamous cell carcinoma have their own unique cancer cell properties and elicit different responses to their respective standard treatment protocols. Investigation of the molecular and cellular properties of the EpCAM-positive and marker-negative cells should provide more insight for the different responses between CRC and HNSCC.

In conclusion, PowerMag system is effective for leukocyte depletion and CTC isolation. Both EpCAM⁺-CTCs and marker-negative cells can be monitored simultaneously that offers benefits in understanding the status of cancer progression and treatment response of cancer patients. The enriched CTCs are viable with a recovery rate of 46-62%. In addition, PowerMag has the capability to isolate rare amounts of CTCs from blood, even at the concentrations of 1-10 CTCs/ml. PowerMag has been demonstrated to recover CTCs from the patients with various cancer types including HNSCC, colorectal cancer, esophageal cancer and gastric cancer. Thus, PowerMag holds great promise as a platform for CTC analysis in a clinical setting.

### Example 2

### Prognostic Value of Circulating Tumor Cells with Podoplanin Expression in Patients with Locally Advanced or Metastatic Head and Neck Squamous Cell Carcinoma

Head and neck squamous cell carcinoma (HNSCC) is a common malignant disease worldwide. The 5-year survival rate for all stages of HNSCC is approximately 40-50%. In locally advanced diseases, which account for about 60% of all HNSCC settings, 20-30% of the patients eventually relapse despite aggressive protocols in the standard first-line treatment such as the combinations of curative surgery, adjuvant radiotherapy or chemoradiotherapy (CRT) and definitive concurrent chemoradiotherapy (CCRT). Even in survival after treatment, the patients are usually impaired in social, speaking and eating abilities. Early detection of recurrence by any examination is thereby crucial for HNSCC patient care. A number of clinical features of HNSCC patients such as TNM staging, comorbidity, sites of origin, treatment modality, and immunohistochemistry staining markers have been proposed for prediction of survivals. New imaging techniques and specialized tests for immunologic responses-associated markers and genetic/epigenetic alteration have also been developed.

Circulating tumor cells (CTCs) are an emerging “liquid biopsy” that provide prognostic value for various types of solid cancer. However, the clinical significance of CTCs in HNSCC is still under debate. Only few studies have been reported and limited conclusions for the prognostic value of CTCs in HNSCC are reached. Moreover, it is clear from several studies that the number of CTCs alone does not provide sufficient information for assessing the status of cancer patients. Protein expression related to epithelial-mesenchymal transition (EMT) in CTCs has been shown to provide add-on values when the clinical status of cancer patients was assessed.

PDPN is a transmembrane protein that has multiple functions in lymphatic vessel formation and cellular cytoskeleton remodeling. PDPN is involved in disease progression of various cancer types by mediating tumor cell-platelet interactions and promoting tumor cell survival in bloodstream. In HNSCC, PDPN has been demonstrated as a novel biomarker for cancer migration/invasion, early lymphatic dissemination, poor prognosis and low response to neoadjuvant chemoradiotherapy. However, whether PDPN is expressed in CTCs from the HNSCC patients and whether PDPN⁺-CTCs have prognostic value have not yet been elucidated. In this study, we address these issues by analyzing the baseline CTCs from 53 locally advanced and metastatic HNSCC patients prior to chemotherapy. We disclose herein for the first time that PDPN is expressed in the CTCs from HNSCC patients. In addition, the ratio of CTCs with PDPN expression but not the absolute CTCs count is a predictive factor of 6-months survival after chemotherapy and a prognostic factor for survival in HNSCC. The significance of these findings is discussed.

### Methods

### Study Design

This was a prospective single-center study approved by the Institute Review Board of Chang Gung Memorial Hospital with the approval ID of 100-4644A3, 100-4623C and 101-2161C. Locally advanced or recurrent/metastasized HNSCC patients that were diagnosed and confirmed histologically or cytopathologically at initial presentation were invited to enroll in this trial with written informed consent. Other criteria of enrollment included (1) age ≧20 years old; (2) adequate liver and renal function and white blood cell counts for anti-cancer chemotherapy; (3) no synchronous cancer or prior cancer history within the last 5 years. Management and the corresponding treatment for the patients at different disease stages were performed according to the institutional guidelines and standard treatment protocols. Enrolled patients were classified into three distinct treatment subgroups: (1) group A: definitive CCRT for the patients with advanced disease; (2) group B: curative surgery followed by adjuvant CRT for the patients with pathologic features such as positive margin (none in this study), pN2, and extracapsular spread (ECS) of involved lymph nodes indicating early relapse according to recommendation from NCCN guidelines; and (3) group C: palliative chemotherapy for the patients with distant metastasis or with poor general condition for definitive CCRT. Blood samples were collected within 7 days prior to the first dose of chemotherapy. The 6-months survival after chemotherapy, progression-free survival (PFS) and overall survival (OS) were used as the endpoints for this study. Study results were reported following REMARK recommendations.

### Enrichment and Isolation of CTCs

Enrichment and isolation of CTCs were performed using the PowerMag system as described above.

### Immunofluorescence Staining and CTCs Enumeration

For immunofluorescence staining, the cell filtrate after depletion of CD45⁺ cells was incubated with anti-EpCAM antibody, anti-PDPN antibody and the Hoechst 33342 DNA staining dye at room temperature for 1 h. According to our previous study, the use of second epithelial cell marker pan-cytokeratin did not provide add-on benefit for CTCs identification and enumeration in our system. Hence, immunofluorescence staining using anti-pan-CK antibody was not necessary and was not performed in this study. After several washes and centrifugation to remove the supernatant, Alexa Fluor 488-conjugated donkey anti-mouse antibody and the Alexa Fluor 555-conjugated goat anti-rat antibody were added to the cell suspension and kept in the dark for 30 min. After removing the unbound antibody, immunofluorescent images were captured by fluorescent microscopy (Zeiss Axiovert 200M). CTCs were defined as the cells that were positive for Hochest 33342 and EpCAM. As shown in our previous report, repeated depletion of CD45⁺ cells by PowerMag column removed almost all of the CD45⁺ cells with only few CD45⁺ cells (<5 cells/ml) in the cell filtrate. Notably, none of these cells was both CD45 and EpCAM positive.

### Single Cell Real-Time PCR Analysis

The EpCAM⁺ cells were picked individually under fluorescent microscopy, and single cell real-time RT-PCR was performed according to the protocol provided by Fluidigm Corporation (South San Francisco, Calif., USA, the relevant portion of the instruction insert is incorporated herein by reference). The expression of PDPN and the internal control genes â-actin and glyceraldehyde 3-phosphate dehydrogenase (GAPDH) was determined using the BioMark HD™ System.

### Statistical Analysis

The number of CTCs in healthy donors and HNSCC patients was compared using boxplot and Kruskal-Wallis test for all groups and Mann-Whitney U test between any two groups with 2-sided significance. The impacts of baseline CTCs number, PDPN⁺-CTCs number, the ratio for the number of PDPN⁺-CTCs over total CTCs (PDPN⁺/EpCAM⁺-CTCs ratio) and factors reported to shorten or prolong survival such as age, body mass index (BMI), performance status (PS) and distant metastasis at initial presentation on PFS and OS were assessed by univariate Cox proportional hazards regression analysis. Parameters with significance in univariate analysis were subjected to multivariate Cox regression analysis. PFS and OS were measured from the date of baseline CTC to the date of confirmed clinical progression, death, or censoring at the last follow-up. Statistical analysis was performed using SPSS for Windows (version 18, SPSS, Chicago, Ill.). A P value of 0.05 was considered statistically significant.

### Results

PDPN was expressed in a subset of CTCs. Both EpCAM⁺-CTCs and PDPN⁺-CTCs counts were statistically different between disease and non-disease groups (P<0.0001) with no prognostic value. After a median follow-up of 10.5 (6.6-18.5) months, the PDPN⁺/EpCAM⁺-CTCs ratio>20% was a significant predictor for 6-months death after chemotherapy (P=0.011) and was correlated with shortened PFS (P=0.016) and OS (P=0.015).

### Conclusions

PDPN⁺/EpCAM⁺-CTCs ratio is a prognostic factor in HNSCC. Defining the ratio in HNSCC patients might be valuable to clinical management.

### Example 3

### Early Detection of Infiltrative Papillary Thyroid Microcarcinoma by Marked Elevated Circulating Tumor Cells Counts

The annual incidence of thyroid cancer is increased 2.4-fold over the past decade which is the most significant increase among different cancer types worldwide. Both the advance of diagnostic tools and the rise in disease occurrence contribute to the elevated incidence of thyroid cancer. In clinical setting, more than 90% of the thyroid cancer belongs to the papillary and follicular thyroid carcinoma. Papillary thyroid microcarcinoma (PTMC) is defined as tumor size equal to or less than 1.0 cm. Most of the PTMC patients have no obvious clinical symptoms and are usually identified during medical checkup or when specific medical procedures are performed. Despite PTMC is usually associated with good prognosis, lymph node metastases and loco-regional recurrences have been reported. While distant metastases and cancer-related death are rare in PTMC patients, it remains controversial whether follow-up treatment is required after thyroidectomy.

In clinical practice, patients with thyroid nodule(s) and normal/elevated levels of serum thyroid stimulating hormone (TSH) are suggested to undergo ultrasound-guided fine-needle aspiration for cytopathological analysis. However, the result is highly dependent on the technical skills of the person who performs the aspiration. Inadequate sampling may result in unsatisfactory sensitivity and accuracy. In order to confirm the status of malignancy, repeated aspiration is recommended to overcome the technical hurdles. Considerable efforts have also been explored to identify other reliable biomarkers for diagnosis of thyroid cancer. Accordingly, new methods for detection of circulating microRNA, specific mutations or circulating tumor cells (CTCs) have been developed to provide better diagnostics or serve as prognostic/predictive factors. Among these methods, CTCs detection is likely a promising approach to fulfill the task.

Herein, we present a case in which the individual was initially enrolled in our CTCs clinical trial as a healthy donor but her abnormally high circulating epithelial cells/CTCs count led to an ultimate diagnosis of PTMC. Interestingly, whole body cancer imaging survey cannot identify malignancy in this patient with certainty. The correlation between CTCs counts and clinical presentation was revealed by series of CTCs testing during the course of post-thyroidectomy anti-thyroid cancer treatment. This example demonstrates the value of CTCs testing as a cancer screening and treatment effectiveness monitoring tool to supplement the currently available medical tests for early cancer detection.

### Case Presentation

An asymptomatic 29 year-old female without any past medical history was invited to undergo survey of clinical trial as a donor in the healthy control group (CTCHNSCC01, NCT01884129, registered on the “clinicaltrial.gov” website). In this trial, CTCs were enriched by the PowerMag negative selection method described herein. A high CTCs (EpCAM⁺ cells) count of 510.0 cells/ml was found to be present in the blood of the donor. To rule out any possible false positive arisen from technical issue in CTCs isolation and detection, the blood from the donor was drawn at a 2-week interval for a second CTCs testing. At the same time, several serum tumor markers including CEA, CA15-3, CA-125, CA19-9 and SCC were examined to define the possible origin of cancer if malignancy was diagnosed. The CTCs (EpCAM⁺ cells) count for the second testing remained high at 470.0 cells/ml. All serum tumor markers (CEA: <0.50 ng/ml [reference: <5]; CA15-3: 9.3 U/ml [reference: <30]; CA-125: 27.8 U/ml [reference: <35]; CA19-9: 9.07 U/ml [reference: <37]; SCC: 1.30 ng/ml [reference: <2.5]) were normal.

Whole body magnetic resonance imaging (MRI) was arranged for tumor survey but no evidence of malignancy could be identified. Moreover, positron emission tomography (PET) scan revealed mild uptake in the left lower thyroid with a standardized uptake value (SUV) of 2.8. The diagnosis of malignancy cannot be made according to the imaging results. With the mild uptake in left thyroid gland, ultrasound was performed to define any thyroid lesion(s). Multiple thyroid nodules with abnormal vascularity were noted. Fine needle aspiration cytology study further concluded that the left thyroid lesion was classified as category III according to the Bethesda cytopathology classification, whereas colloid with a few benign follicular cells over right side thyroid gland as category II. Initial serum anti-thyroglobulin (Tg) antibody (ATA) test was negative. Because the suspicion for thyroid malignancy keeps rising even though no definite evidence of thyroid cancer was obtained, thyroidectomy was decided after full discussion. Final pathology report revealed an infiltrative PTMC in the left thyroid gland.

Two weeks after total thyroidectomy, CTCs and Tg tests were performed before thyrogen and remnant ablation treatment using the radioactive iodine (¹³¹I). CTCs count dropped sharply to 214.0 cells/ml with a Tg level of 6.26 ng/ml. Serum Tg was determined by either the Tg-immunoradiometric assay Tg-IRMA (Cis Bio International, Gif-sur-Yvette, France) or a highly sensitive chemiluminescent assay Tg Access with detection limit <0.1 ng/ml (Beckman Coulter, Fullerton, Calif.). The enriched CTCs population was also analyzed by immunofluorescence staining of thyroid stimulating hormone receptor (TSHR). The positive TSHR staining signal confirmed thyroid-related tissue as the origin of CTCs. Whole body cancer workup at 6 days after remnant ablation treatment using 1.1 GBq (30 mCi) of ¹³¹I revealed multiple focal areas with increased uptake of radioactivity. These focal areas were solely found in the neck region but not in other organ sites.

Both CTCs and Tg tests were performed to monitor disease status during the subsequent follow-up. Series of follow-up CTCs counts showed high correlation between the patient's clinical course presentation and CTCs enumeration. At the 1.5^{th} month after surgery, Tg was undetectable (<1.2 ng/ml by Tg-IRMA), while CTCs count showed a marked decrease (31.1 cells/ml) but was not yet within the normal range which was defined as less than 5 cells/ml (18). Another follow-up at 3^{rd} month after surgery showed a decreasing trend of CTCs count (24.5 cells/ml) and the accompanying Tg are within normal range (<1.2 ng/ml by Tg-IRMA). At the 8^{th} month after thyroidectomy, a low and near-normal CTCs count (5.5 cells/ml) and Tg<0.1 ng/ml (Tg Access) were achieved. Recently, the patient underwent 2 mCi radioactive iodide thyroid scan. An uptake of <0.4% with a low CTC count of 7.3 cells/ml was achieved at the 10^{th} month after surgery. At this stage, the patient has reached a disease-free status and is still under close monitoring for CTCs count and Tg level at outpatient department by endocrinologist and medical oncologist.

### Methods

CTCs isolation and enumeration were performed by PowerMag as described above. Tg was measured using Tg-IRMA (Cis Bio International) or a highly sensitive assay Tg Access (Beckman Coulter).

### Results

In this case, cancer diagnosis could not be confirmed by MRI, PET and cytopathological study. Lesions in the left lobe of thyroid were found by thyroid gland ultrasound and were histologically confirmed as PTMC. Both CTCs counts and Tg were significantly decreased during the treatment course. Notably, CTCs was sustained and detectable when Tg was normal at 1.5-5.5 months after thyroidectomy. The number of CTCs returned to normal and the patient reached disease-free at 10 months after thyroidectomy.

### Conclusions

CTCs testing could be used to identify individuals at risk of cancer. CTCs might also complement Tg testing and provide important information for thyroid cancer patient care.

TABLE 1
The efficiency of PowerMag and EasySep in
leukocyte depletion and CTC recovery.

| | PowerMag | EasySep | PowerMag | EasySep |
|---|---|---|---|---|
| Times of | (10⁷ leukocytes) | | (10⁶ leukocytes) | |
| depletion | No. of leukocytes retained (mean ± SE) | | | |
| 1 | 32,013 ± 2,520** | 272,280 ± 46,899 | 1,552 ± 171* | 31,938 ± 7,960 |
| 2 | 325 ± 39* | 19,382 ± 4,782 | 124 ± 37* | 1,089 ± 307 |
| 3 | 95 ± 12* | 1,967 ± 567 | 66 ± 2** | 513 ± 59 |
| 4 | 20 ± 2** | 126 ± 16 | 10 ± 1** | 103 ± 9 |

| | PowerMag | EasySep | PowerMag | EasySep |
|---|---|---|---|---|
| Times of | (100 PC3 cells in 10⁷ leukocytes) | | (100 PC3 cells is in 10⁶ leukocytes) | |
| depletion | Recovery rate of PC3 cells (%, mean ± SE) | | | |
| 1 | 101.8 ± 2.9** | 75.8 ± 2.7 | 103.3 ± 2.7** | 80.4 ± 3.2 |
| 2 | 93.7 ± 3.8** | 61.4 ± 2.2 | 90.5 ± 2.4** | 71.5 ± 2.9 |
| 3 | 83.5 ± 4.8** | 48.3 ± 3.0 | 88.9 ± 2.6* | 61.1 ± 7.87 |
| 4 | 77.7 ± 2.7*** | 39.0 ± 2.9 | 78.4 ± 3.6* | 48.4 ± 5.9 |

*p < 0.05.
**p < 0.01.
***p < 0.001.

TABLE 2
The number of PC3 cancer cells recovered from spiking test

| | Spiking test | | | | | | | | | | |
|---|---|---|---|---|---|---|---|---|---|---|---|
| No. of | 1 | 2 | 3 | 4 | 5 | 6 | 7 | 8 | 9 | 10 | |
| cell spiked | No. of cell recovery | | | | | | | | | | Mean ± SD |
| 1 | 1 | 0 | 1 | 0 | 0 | 1 | 1 | 1 | 0 | 1 | — |
| 5 | 2 | 5 | 2 | 3 | 3 | 3 | 3 | 3 | 3 | 3 | 3.0 ± 0.3 |
| 10 | 7 | 6 | 8 | 6 | 8 | 6 | 5 | 6 | 7 | 8 | 6.7 ± 0.3 |

TABLE 3
Basic characteristics for the cancer
patients subject to CTC analyses.

| Patient characteristics | CRC^{a} | HNSCC^{a} |
|---|---|---|
| Number of patients | 24 | 28 |
| Median age (range), years | 61.5 (38-82) | 56 (41-80) |
| Sex (male/female) | 13/11 | 26/2 |
| Stage III/IV without distant metastasis^{b} | 4 | 21 |
| Stage IV with distant metastasis | 20 | 7 |

^{a}CRC, colorectal carcinoma; HNSCC: head and neck squamous cell carcinoma.
^{b}According to American Joint Committee on Cancer (AJCC), 7th edition, 2010. In HNSCC, stage IVa or IVb means locally advanced stage (often extended regional lymph node involvement without distant metastasis).

TABLE 4
CTC status for healthy control and cancer patients.

| | | EpCAM⁺ | | | Markers⁻ | | | EpCAM⁺ ≧5 or |
|---|---|---|---|---|---|---|---|---|
| Type | Condition | Median | Mean | ≧5 | Median | Mean | ≧650 | markers⁻ ≧650 |
| Normal | Control (n = 27) | 1.3 | 1.6 | 0.00% | 182 | 250.8 | 11.10% | 11.10% |
| CRC | Baseline (n = 24) | 56.3 | 461.7 | 83.30% | 755.9 | 1411.2 | 61.10% | 95.80% |
| | Follow-up (n = 22) | 7.2 | 17.7 | 54.50% | 519 | 1019.3 | 45.50% | 77.30% |
| | Change (%) | −87.3 | −96.2 | — | −24.2 | −25.7 | — | — |
| HNSCC | Baseline (n = 28) | 24.8 | 109.4 | 100.00% | 889.4 | 1347.3 | 60.70% | 100.00% |
| | Follow-up (n = 19) | 21 | 76.3 | 94.70% | 371 | 760.7 | 47.40% | 94.70% |
| | Change (%) | −15.2 | −30.2 | — | −58.3 | −43.5 | — | — |

What is claimed is:

1. A method for isolating and enriching non-leukocyte nucleated cells from a blood sample, comprising:
removing erythrocytes from the blood sample to form a nucleated cell-suspension in a cell culture medium;
labeling leukocytes in the nucleated cell-suspension with magnetic nanoparticles;
loading the cell suspension onto a separation column to yield a filtrate with diminished or depleted leukocytes, wherein:
said separation column comprising:
a body with an entry end and an exit end each having an opening disposed thereon, and
a cylindrical hollow space connecting the openings at the entry end and the exit end to form a passage channel;
said passage channel is packed with spherical separation beads having a uniform size in the range from about 0.5 mm to about 1.5 mm and evenly distributed to fill up the channel,
said separation beads are capable of being magnetized and are coated with an anti-corrosion coating, and
said column is placed in a circular magnetic field;
collecting the filtrate; and
repeating the labeling, loading, and collecting steps above for a predetermined number of cycles or until the amount of leukocyte in the filtrate is below a predetermined level, each cycle using the filtrate of the previous cycle as the nucleated cell-suspension for the labeling step.

2. The method of claim 1, wherein said labeling is accomplished by a tetrameric antibody complex directed against leukocyte-specific surface antigen tethered to magnetic nanoparticles having a uniform size of about 200 nm, said nanoparticles are coated with dextran.

3. The method of claim 1, wherein said column further comprises a barrier disposed at the exit end, said barrier is configured such that no separation bead is allowed to exit but cells and medium are allowed to flow through.

4. The method of claim 1, wherein said separation beads are iron beads treated with a nickel coating or any anti-corrosion materials, said separations beads are packed in the column to a density of about 963 beads/cm³.

5. The method of claim 4, wherein said column further include a barrier disposed at the exit end of the body, said barrier is configured to retain the bead in the column while allowing fluids and cells to flow through.

6. The method of claim 1, wherein said cell culture medium is nutrition medium in the presence of FBS, RPMI in the presence of 20% FBS.

7. The method of claim 1, wherein said separation beads are about 1.0 mm in size, and the predetermined number of cycles is between 2-4 cycles.

8. A system for isolating and enriching non-leukocyte nucleated cells from a blood sample, comprising:
a separation column comprising:
a body with an entry end and an exit end each having an opening disposed thereon, and
a cylindrical hollow space connecting the openings at the entry end and the exit end to form a passage channel;
a plurality of spherical separation beads disposed in the passage channel of the separation column, wherein said separation beads are comprised of a ferromagnetic material coated with an anti-corrosion coating, have a uniform size of about 0.5 mm to about 1.5 mm, and are capable of being magnetized to capture a cell labeled with magnetic nanoparticles; and
a magnet capable of generating a circular magnetic field;
wherein said separation beads are packed in the separation column to fill the passage channel, said magnetic nanoparticles are attached to leukocytes in a blood sample to be passed through the separation column, and said separation column is placed in the circular magnetic field generated by the magnet such that the separation beads are sufficiently magnetized to magnetically capture the nanoparticles.

9. The system of claim 8, wherein said separation beads are spherical iron beads coated with nickel.

10. The system of claim 8, wherein said column further comprising a barrier disposed at the exit en, said barrier is configured such that no separation bead is allowed to exit but cells and medium are allowed to flow through.

11. The system of claim 8 further comprising a plurality of actuators and a computer control unit configured to perform a leukocyte depletion process comprising the steps of:
introducing a nucleated cell-suspension containing leukocytes labeled with the magnetic nanoparticles to the separation column through the entry opening of the column;
allowing the nucleated cell-suspension to flow through the passage channel of the column to yield a filtrate containing diminished or depleted leukocytes;
collecting the filtrate at the exit opening;
labeling the leukocytes remaining in the filtrate with magnetic nanoparticles; and
repeating the above steps for a predetermined number of times.

12. The system of claim 11, wherein the depletion process is repeated for 3-4 times.

13. A method for early detection of cancer in a patient, comprising:
obtaining a blood sample from the patient;
depleting leukocytes in the blood sample to yield a leukocyte-depleted cell-suspension by performing the method of claim 1;
enumerating cells in the leukocyte-depleted suspension that are EpCAM⁻CD45⁻ or EpCAM⁺; and
determining a diagnosis wherein if the amount of EpCAM⁻CD45⁻ cells is ≧650 cells/ml or the amount of EpCAM+ cells is ≧5 cells/ml, the patient is determined to be at high risk of having a cancer, otherwise, the patient is determined to be at low risk of having a cancer.

14. A method for monitoring or assessing the effectiveness of a cancer treatment on a patient, comprising:
obtaining a first blood sample of the patient prior to the cancer treatment and establishing a baseline circulating tumor cell (CTC) count by depleting leukocytes from the sample using the method of claim 1 and enumerating a CTC count, wherein CTC count is defined as the amount of EpCAM⁺ cells and EpCAM⁻CD45⁻ cells in the leukocyte-depleted blood sample;
obtaining a second blood sample of the patient after the cancer treatment and determining a post-treatment level of CTC count by depleting leukocytes from the sample using the method of claim 1 and enumerating a CTC count; and
comparing the levels of post-treatment CTC count to the baseline CTC count, and optionally obtaining additional blood samples at different time intervals after the cancer treatment to determine a time-series for post-treatment CTC counts, wherein if the post-treatment CTC counts show a decreasing trend, the treatment is said to be effective, whereas if the post-treatment CTC count shows an increasing trend or stays at about the baseline level, the treatment is said to be ineffective.

15. A circulating tumor cell (CTC)-based assay for determining a prognosis of a patient suffering from head and neck squamous cell carcinoma or other types of carcinomas, comprising:
obtaining a blood sample from the patient;
depleting leukocytes from the blood sample to yield a leukocyte-depleted cell-suspension by applying the method of claim 1 to the blood sample;
enumerating CTC count in the leukocyte-depleted cell-suspension, wherein CTCs are defined as the cells that are positive for PDPN and/or EpCAM expression;
computing a ratio for the number of CTC expressing PDPN to the total number of CTC; and
determining a prognosis for the patient based on the ratio, wherein if the ratio is greater than 20%, said patient is said to have a poor prognosis for 6-months survival after chemotherapy.

16. A circulating tumor cell (CTC)-based assay for early detection of infiltrative papillary thyroid microcarcinoma (PTMC) in a patient, comprising:
obtaining a blood sample from the patient;
depleting leukocytes from the blood sample to yield a leukocyte-depleted cell-suspension by applying the method of claim 1 to the blood sample;
enumerating a CTC count in the leukocyte-depleted cell-suspension, wherein CTC is defined as EpCAM⁺ cells; and
determining a diagnosis based on the CTC count, wherein if the CTC count is above a predetermined level, a likelihood of PTMC is indicated.

17. The method of claim 18, wherein said predetermined level of CTC count is selected from 5 cells/ml, 200 cells/ml, and 500 cells/ml.

18. A kit for isolating and enriching CD45⁻-nucleated cells in a blood sample, comprising:
a red blood cell (RBC) lysis reagent;
CD45 depletion cocktails and reagents;
a dextran-coated magnetic nanoparticles having an uniform size of about 200 nm;
a cell culture or a nutrition medium; and
an instruction insert having encoded thereon a human readable description of the method of claim 1.

19. The kit of claim 18, further comprising a separation column, wherein said column comprising:
a body with an entry end and an exit end each having an opening disposed thereon; and
a cylindrical hollow space connecting the openings at the entry end and the exit end to form a passage channel,
wherein said column is pre-packaged with a plurality of spherical separation beads disposed in the passage channel, said separation beads are comprised of a ferromagnetic material coated with an anti-corrosion coating, have a uniform size of about 0.5 mm to about 1.5 mm, and are capable of being magnetized to capture a cell labeled with magnetic nanoparticles.

20. The kit of claim 18, further comprising fluorescent staining reagents and antibodies for cancer cell markers.
